(19) United States
(12) Patent Application Publication
(10) Pub. No.: US 2007/0282252 A1
(43) Pub. Date: Dec. 6, 2007

(54) Method for oral delivery of a healing substance to a target place in gastrointestinal tract of human or animals

(76) Inventor: Igor Igorevich Stukanov, Toronto (CA)

Correspondence Address: IGOR STUKANOV, SUITE 405, 66 OAKMOUNT ROAD, TORONTO, ON, M6P 2M8, CA

(21) Appl. No.: 11/650,556

(22) Filed: Jan. 8, 2007

Related U.S. Application Data
(60) Provisional application No. 60/776,908, filed on Feb. 24, 2006.

(51) Int. Cl. A61M 31/00

(52) U.S. Cl. 604/93.01

(57) ABSTRACT

A method for oral delivery of new drugs, big molecules, proteins, and healing substances to a target place in the body, via digestive tract, using currently available technologies of mobile wireless controlled capsules with sensors.

### CROSS-REFERENCE TO RELATED APPLICATIONS

This application claims priority status of provisional patent application U.S. 60/776,908 filed Feb. 24, 2006

### STATEMENT REGARDING FEDERALLY SPONSORED RESEARCH OR DEVELOPMENT

Not Applicable

### REFERENCE TO A MICROFICHE APPENDIX

Not Applicable

### BACKGROUND OF THE INVENTION

1. Field of the Invention

This invention relates to oral controlled high precision drug and healing substances/agents delivery methods in humans and animals.

2. Background Information

The field of advanced drug delivery is more than thirty years old. There are two main problems with oral delivering of healing substances such as proteins and biotech bio-molecules. The first problem is in the destruction of big healing substances such as proteins, peptides or big molecules by the agents in the digestive systems if these healing substances were taken orally. The second problem is in inaccuracy of targeting damaged regions. For example, chemotherapy may damage non-cancerous cells, because there is no way to detect with high precision cancerous cells inside the gastrointestinal tract by current chemical therapy and radiological therapy methods.

More than thousands of methods were proposed for solutions of these problems, which include the usage of electric field, and/or magnetic field, and/or ultrasound, and/or light, and/or enzymes, and/or viruses, and/or temperature, and/or pH, and/or mico-sized valves/chambers/pumps, and/or smart-microchips, and/or biopolymers, and/or nanoparticles, etc. Although some of these delivery technologies may be effective they are not cost efficient today and usually are restricted to the specific drugs, which can be delivered by these methods.

The purpose of the current invention is to suggest a reliable, low-cost, non-restricted to particular drugs, and convenient method for oral delivery of new drugs, big molecules, proteins, and other healing substances to the targeted place in the body via digestive tract, using currently available technologies of mobile wireless controlled capsules with sensors.

### BRIEF SUMMARY OF THE INVENTION

The current invention consists in using commercially available wireless controlled capsules with imaging sensors to deliver healing substances to the target place in the gastro-digestive tract. Wireless controlled capsules with imaging sensors, such as PillCam (Given Imaging, Inc.) or SmartPills (Smart Pill Corporation) allow to deliver healing substances to a targeted region with very high precision, which allow to solve the problem of high precision targeting of damaged regions and to avoid exposure of non-damaged regions to healing substances. The second advantage of the current invention consist in delivery of peptides, proteins, and other big molecular structures, which are normally decomposed in the digestive tract if taken orally, into blood stream without their exposure to digestive machinery and without modifications in their structure and function.

### BRIEF DESCRIPTION OF THE SEVERAL VIEWS OF THE DRAWINGS

Not applicable

### DETAILED DESCRIPTION OF THE INVENTION

The present invention is directed to a method for oral delivery of new drugs, big molecules, proteins, healing substances to a target place in the body, via digestive tract, using currently available technologies of mobile wireless controlled capsules with sensors.

Wireless mobile capsules, for example such as PillCam manufactured by Given Imaging, Inc. or SmartPills manufactured by SmartPill Corporation allow to monitor digestive tract in real-time via imaging sensors, wireless communication, and control system with very high precision, convenience for patients, and with low costs.

In this capsule is embedded healing substance delivery mechanism, which may be any standard mechanism used today, for example, injectors, micro guns, pushers, pumps, etc. This mechanism is activated by an operator (human or computer system) via wireless communication channel.

Wireless controlled capsules with imaging sensors allow to deliver healing substances to a targeted region with very high precision, which allow to solve the problem of high precision targeting of damaged regions and avoiding exposure of non-damaged regions to healing substances.

When the operator, which may be a human or computer system, see via imaging system that the capsule is close enough to the targeted region, the operator send signal to the capsule to deliver the healing substance via the embedded healing substance delivery mechanism. This mechanism directs the healing substance to the targeted place.

The invention is described below on several examples.

### EXAMPLE 1

### Cancer in Gastrointestinal Tract

The delivery part of mobile wireless controlled capsule is loaded with a healing substance, for example, molecules with special receptors with attached chemical or radioactive agent to treat cancerous cells. A patient swallow the capsule, which send images of gastrointestinal tract to an operator as it travels in the tract. The operator (human or computer system) analyzes the images sent by the capsule. When the operator detect that the capsule is close enough to the target, the signal is sent to the capsule to direct the healing substance to the target. This command is executed by capsule healing substance delivery mechanism, which may be any standard mechanism used today, for example, injectors, micro guns, pushers, pumps, etc. The healing substance directed to the target region, sticks to the target, and treats the cancer.

### EXAMPLE 2

### Delivering proteins or peptides into the blood system

The delivery part of mobile wireless controlled capsule is loaded with proteins or peptides. A patient swallow the capsule, which send images of gastrointestinal tract to an operator as it travels in the tract. The operator (human or computer system) analyzes the images sent by the capsule. When the operator detect that the capsule passed the region where the proteins or peptides are destroyed by digestive enzymes in the gastrointestinal tract, the signal is sent to the capsule to release the proteins or peptides. This command is executed by capsule healing substance delivery mechanism, which release the proteins or peptides. These proteins or peptides then flow undamaged into the blood system.

The advantage of the current invention consist in delivery of peptides, proteins, and other big molecular structures, which are normally decomposed in digestive tract if taken orally, into blood stream without their exposure to digestive machinery and without modifications in their structure and function.

The next advantage of the current invention is the high accuracy of targeting the damaged regions.

The next advantage of the current invention is high degree of safety for using this method.

1. A method for delivery of a healing substance to a target place in gastrointestinal tract or blood system, comprising the following steps:
a). A healing substance is placed into a mobile wireless controlled capsule with sensors.
b). The capsule enters into the gastrointestinal tract via mouth of a patient with food or water and moves toward the targeted place.
c). The capsule is directed to the targeted place via control signals sent from a control unit wirelessly. The capsule send signals from the sensors to a monitor connected to the control unit.
d). When the capsule reach the targeted place the control unit send signal to release the healing substance and this healing substance is released from the capsule and directed to the targeted place via signals from the control unit.

2. The method as in claim 1, where the healing substance is a composition of chemical elements in solid or fluid or gases form.

3. The method as in claim 1, where the healing substance is a set of electromagnetic waves.

4. The method as in claim 1, where the healing substance is a set of mechanical waves.

5. The method as in claim 1, where the healing substance is a set of sound or ultrasound waves.

6. The method as in claim 1, where the healing substance is a flow of physical particles.

7. The method as in claim 1, where the signal to release the healing substance is sent by a human operator.

8. The method as in claim 1, where the signal to release the healing substance is sent by the control unit automatically.
